SEQUENCE LISTING (5 sequences)

SEQ ID NO: 1 - protein, 210 aa, Homo sapiens
QSALTQPPSASGSPGQSVTISCTGTSSDVGGYNYVSWYQHHPGKAPKLIISEVNNRPSGVPDRFSGSKSGNTASLTVSGLQAEDEAEYYCSSYTDIHNFVFGGGTKVTVLGQPKANPTVTLFPPSSEELQANKATLVCLISDFYPGAVTVAWKADSSPVKAGVETTTPSKQSNNKYAASSYLSLTPEQWKSHKSYSCQVTHEGSTVEKTV

SEQ ID NO: 2 - DNA, 630 bp, Homo sapiens
cagtctgccctgactcagcctccctccgcgtccgggtctcctggacagtcagtcaccatctcctgcactggaaccagcagtgacgttggtggttataactatgtttcctggtaccaacaccacccaggcaaagcccccaaactcataatttctgaggtcaataaccggccctcaggggtccctgatcgtttctctggctccaagtctggcaacacggcctccctgaccgtctctgggctccaggctgaggatgaggctgaatattactgcagctcatacacagacatccacaatttcgtcttcggcggagggaccaaggtcaccgtcctaggtcagcccaaggccaaccccactgtcactctgttcccgccctcctctgaggaactgcaagccaacaaggccactctggtgtgtctgatcagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtg

SEQ ID NO: 3 - protein, 230 aa, Homo sapiens
QVQLQQWGPGLVKPSQTLSLSCTVSGGSSSSGAHYWSWIRQYPGKGLEWIGYIHYSGNTYYNPSLKSRITISQHTSENQFSLKLNSVTVADTAVYYCARGTRLRTLRNAFDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTS

SEQ ID NO: 4 - DNA, 702 bp, Homo sapiens
caggtgcagctacagcagtggggcccaggactggtgaagccttcacagaccttgtccctcagttgcactgtctctggtggctccagcagtagtggtgctcactactggagttggatccgccagtacccagggaagggcctggagtggattggttacatccattacagtgggaacacttactacaacccgtccctcaagagtcgaattaccatatcacaacacacgtctgagaaccagttctccctgaagctcaactctgtgactgttgcagacacggccgtctattactgtgcgagagggacccgtctccggacactacggaatgcttttgatatttggggccaggggaccacggtcaccgtctcctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactagctaattaatttaa

SEQ ID NO: 5 - protein, 584 aa, Artificial
  Synthetic
MKYLLPTAAAGLLLLAAQPAMAQVQLQQWGPGLVKPSQTLSLSCTVSGGSSSSGAHYWSWIRQYPGKGLEWIGYIHYSGNTYYNPSLKSRITISQHTSENQFSLKLNSVTVADTAVYYCARGTRLRTLRNAFDIWGQGTTVTVSSGGGGSGGGGSGGGGSQSALTQPPSASGSPGQSVTISCTGTSSDVGGYNYVSWYQHHPGKAPKLIISEVNNRPSGVPDRFSGSKSGNTASLTVSGLQAEDEAEYYCSSYTDIHNFVFGGGTKVTVLGQPKANTSGGSGGGSGGGSGGGSGGGSGGGSGELVLTQSPGTLSLSAGERATLSCRASQSVSSGSLAWYQQKPGQAPRLLIYGASTRATGIPDRFSGSGSGTDFTLTIGRLEPEDLAVYYCQQYGTSPYTFGQGTKLEIKRTGGGGSGGGGSGGGGSVQLLEQSGAEVKKPGSSVQVSCKASGGTFSMYGFNWVRQAPGHGLEWMGGIIPIFGTTNYAQKFRGRVTFTADQATSTAYMELTNLRSDDTAVYYCARDFGPDWEGGDSYDGSGRGFFDFWGQGTLVNVSSAAAHHHHHHGAAEQKLISEEDLNGAA

(19) United States
(12) Patent Application Publication
(10) Pub. No.: US 2008/0038280 A1
(43) Pub. Date: Feb. 14, 2008

(54) A32 Monoclonal Antibody Fusion Proteins For Use As Hiv Inhibitors And Vaccines

(75) Inventors: Dimiter S. Dimitrov, Frederick, MD (US); Mei-Yun Zhang, Frederick, MD (US)

Correspondence Address: LEYDIG, VOIT & MAYER, LTD., TWO PRUDENTIAL PLAZA, SUITE 4900, 180 NORTH STETSON AVENUE, CHICAGO, IL, 60601-6731, US

(73) Assignee: Government of the United States of America, Rockville, MD (US)

(21) Appl. No.: 11/577,237

(22) PCT Filed: Oct. 11, 2005
(86) PCT No.: PCT/US05/36568

§ 371 Date: Apr. 26, 2007

Related U.S. Application Data
(60) Provisional application No. 60/618,820, filed on Oct. 14, 2004.

(51) Int. Cl. A61K 39/395; A61K 31/711; A61P 31/18; C07H 21/04; C07K 16/08; C12N 5/06

(52) U.S. Cl. 424/178.1; 435/325; 514/44; 530/387.3; 536/23.4

(57) ABSTRACT

The invention provides a fusion protein, which comprises an antigen binding portion of an A32 human antibody, or variant thereof, and one of the following: (a) an antigen-binding portion of a second antibody or variant thereof, wherein the second antibody binds to an epitope of an envelope protein of a human immunodeficiency virus (HIV) that is exposed upon the HIV binding to a CD4 receptor, (b) an immunogenic portion of an envelope protein of a HIV, or a variant thereof, or (c) a soluble CD4 (sCD4) polypeptide capable of binding to HIV, or a or variant thereof.

### FIELD OF THE INVENTION

This invention pertains to a fusion protein inhibitor of HIV infection and methods of using same.

### BACKGROUND OF THE INVENTION

The Human Immunodeficiency Virus (HIV) is the causative agent of Acquired Immunodeficiency Syndrome (AIDS). HIV type 1 (HIV-1) entry into host cells is initiated by the binding of the gp120 subunit of the viral envelope glycoprotein (Env) complex to the host cell receptor (CD4) (see, e.g., Dalgleish et al., Nature, 312, 763-767 (1984); and Klatzmann et al., Nature, 312, 767-768 (1984)). This interaction induces conformational changes in gp120 resulting in the exposure of a conserved high-affinity binding site for the co-receptor (i.e., the chemokine receptor CCR5 or CXCR4) (see, e.g., Sattentau et al., J. Exp. Med., 174, 407-415 (1991), Sattentau et al., J. Virol., 67, 7383-7393 (1993), Thali et al., J. Virol., 67, 3978-3988 (1993), Trkola et al., J. Virol., 70, 1100-1108 (1996), and Wu et al., Nature, 384, 179-183 (1996)).

Binding of Env to CD4 and either co-receptor initiates a series of conformational changes that lead to viral entry into the target cell. Therefore, efforts to develop a vaccine for the prevention and/or treatment of HIV infection have focused upon the development of neutralizing antibodies that specifically bind to Env. However, the extensive variation of Env in the numerous isolates of HIV has presented a major obstacle in designing an effective immunogen for the isolation of antibodies with broadly neutralizing activity against multiple HIV isolates.

Neutralizing antibodies are believed to act, at least in part, by binding to the exposed Env surface and obstructing the initial interaction between a trimeric array of gp120 molecules on the virion surface and receptor molecules on the target cell (see, e.g., Parren et al., Adv. Immunol., 77, 195-262 (2001); Parren et al., J. Virol., 72, 3512-3519 (1998); and Ugolini et al., J. Exp. Med., 186, 1287-1298 (1997)). HIV-1 has evolved a number of strategies to evade recognition by neutralizing antibodies, particularly those directed to the conserved CD4 and co-receptor binding sites of Env. The extent of protection of these sites from antibody recognition is limited by the necessity to preserve the accessibility for receptor interaction. In the case of the binding site of CD4 (CD4bs), the following structural features have resulted: (i) CD4bs is partially obscured from antibody recognition by the V1/V2 loop and associated carbohydrate structures, (ii) the flanking residues are variable and modified by glycosylation, (iii) CD4bs is recessed to an extent that limits direct access by an antibody variable region, (iv) clusters of residues within the CD4bs that do not directly interact with CD4 are subject to variation among strains, (v) many gp120 residues interact with CD4 via main-chain atoms, allowing for variability in the corresponding amino acid side chains, and (vi) there is considerable conformational flexibility within the CD4-unbound state of gp120. Antibody binding, therefore, requires relatively large entropic decreases, thus conformationally masking the conserved CD4bs (see, e.g., Labrijn et al., J. Virol., 77(19), 10557-10565 (2003)).

The co-receptor binding site of gp120 is thought to be composed of a highly conserved element on the β19 strand and parts of the V3 loop (see, e.g., Rizzuto et al., AIDS Res. Hum. Retrovir., 16, 741-749 (2000); Rizzuto et al., Science, 280, 1949-1953 (1998); and Wyatt et al., Science, 280, 1884-1888 (1998)). These elements are masked by the V1/V2 variable loops in the CD4-unbound state and are largely unavailable for antibody binding (see, e.g., Trkola et al., Nature, 384, 184-187 (1996); and Wu et al., supra). Upon CD4 binding, conformational changes are induced, which include displacement of the V1/V2 stem-loop structure and consequent exposure of the co-receptor-binding site (see, e.g., Moore et al., J. Virol., 67, 6136-6151 (1993), Sattentau et al. (1993), supra, and Wyatt et al., J. Virol., 69, 5723-5733 (1995)). Binding studies with variable loop-deleted mutants suggest that CD4 induces additional rearrangement or stabilization of the gp120 bridging sheet near the β19 strand to form the final co-receptor-binding site (see, e.g., Wu et al., supra; and Wyatt et al. (1998), supra). Since the binding to CD4 occurs at the virus-cell interface, the exposed co-receptor binding site is optimally positioned for interaction with the co-receptor.

A highly conserved discontinuous structure on gp120 associated with the co-receptor binding site is recognized by monoclonal antibodies (mAbs) that bind better to gp120 upon binding with CD4. These CD4-induced (CD4i) antibodies, such as 17b and 48d, recognize a cluster of gp120 epitopes that are centered on the β19 strand and partially overlap the co-receptor binding site (see, e.g., Rizzuto et al. (2000), supra; Rizzuto et al. (1998), supra; Trkola et al. (1998), supra; and Wu et al., supra). Although such CD4i mAbs can neutralize some T-cell line-adapted HIV-1 strains, they are, generally poorly neutralizing for primary isolates because their potency and related ability to suppress the generation of HIV-1 escape mutants are low. Recently, the antibody Fab fragment, X5, was isolated from a phage display library (see, e.g., International Patent Application WO 03/033666). Fab X5 is directed to a CD4i epitope and neutralizes a wide variety of primary isolates (see, e.g., Moulard et al., Proc. Natl. Acad. Sci. USA, 99, 6913-6918 (2002)), although the whole immunoglobulin G of X5, IgG X5, does not have a similar effect (see, e.g., Labrijn et al., supra).

The A32 human monoclonal antibody is a CD4 mimic that also recognizes a discontinuous epitope on gp120 (see, e.g., Boots et al., AIDS Research and Human Retroviruses, 13, 1549-1559 (1997)). A32, however, does not bind gp120 at the CD4 binding site (see, e.g., Wyatt et al., supra). Like CD4, A32 exposes the CCR5 binding site on recombinant gp120, and enhances the binding of CD4i antibodies 17b and 48d (see, e.g., Wyatt et al., supra). A32 itself has not been shown to induce significant neutralization of HIV isolates.

There remains a need for molecules that can neutralize a broad range of HIV-1 isolates, and methods of using such molecules to inhibit HIV-1 infection in a human host. The invention provides such molecules and methods. These and other advantages of the invention, as well as additional inventive features, will be apparent from the description of the invention provided herein.

### BRIEF SUMMARY OF THE INVENTION

The invention provides a fusion protein, which comprises an antigen binding portion of an A32 human antibody, or variant thereof, and one of the following: (a) an antigen-binding portion of a second antibody, or a variant thereof, wherein the second antibody-binds to an epitope of an envelope protein of a human immunodeficiency virus (HIV) that is exposed upon the HIV binding to a CD4 receptor, (b) an immunogenic portion of an envelope protein of a HIV, or a variant thereof, or (c) a soluble CD4 (sCD4) polypeptide capable of binding to HIV, or a or variant thereof.

The invention also provides a fusion protein, which comprises (i) a light chain amino acid sequence of an A32 human antibody, or a variant thereof, or a heavy chain amino acid sequence of an A32 human antibody, or a variant thereof, and (ii) one of the following: (a) an antigen-binding portion of a second antibody, or a variant thereof, wherein the second antibody binds to an epitope of an envelope protein of a human immunodeficiency virus (HIV) that is exposed upon the HIV binding to a CD4 receptor, (b) an immunogenic portion of an envelope protein of a HIV, or a variant thereof, or (c) a soluble CD4 (sCD4) polypeptide capable of binding to HIV, or a or variant thereof.

### DETAILED DESCRIPTION OF THE INVENTION

The invention provides a fusion protein comprising an antigen binding portion of an A32 human antibody, or variant thereof. The invention also provides a fusion protein comprising a light chain amino acid sequence of an A32 human antibody, or a variant thereof, or a heavy chain amino acid sequence of an A32 human antibody, or a variant thereof. As is known in the art, the A32 human antibody is a monoclonal IgG1 immunoglobulin molecule that recognizes a discontinuous epitope on the HIV-1 gp120 envelope protein of most HIV-1 clade B isolates (see, e.g., Boots et al., supra). The fusion protein can comprise any suitable portion of the A32 antibody, so long as the portion can recognize and bind to an appropriate antigen (e.g., a gp120 epitope). In this regard, the fusion protein can comprise the full-length amino acid sequence of the A32 antibody molecule. Alternatively, the antigen binding portion of the A32 antibody preferably comprises a fragment of A32 amino acid sequence. In this respect, proteolytic cleavage of an intact antibody molecule can produce a variety of antibody fragments that retain the ability to recognize and bind antigens. For example, limited digestion of an antibody molecule with the protease papain typically produces three fragments; two of which are identical and are referred to as “Fab” fragments, as they retain the antigen binding activity of the parent antibody molecule. Cleavage of an antibody molecule with the enzyme pepsin produces two antibody fragments, one of which retains both antigen-binding arms of the antibody molecule, and is referred to as the F(ab′)₂ fragment. A single-chain variable region antibody fragment (scFv), which essentially consists of a truncated Fab fragment comprising the variable (V) domain of an antibody heavy chain linked to a V domain of a light antibody chain via a synthetic peptide, can be generated using routine recombinant DNA technology techniques (see, e.g., C. A. Janeway et al. (eds.), Immunobiology, 5^{th} Ed., Garland Publishing, New York, N.Y. (2001)). Similarly, disulfide-stabilized variable region fragments (dsFv) can be prepared by recombinant DNA technology (see, e.g., Reiter et al., Protein Engineering, 7, 697-704 (1994)). Enzymatic cleavage also can product an Fd antibody fragment, which contains the N-terminal half of the heavy chain of the antibody. The antigen binding portion of the A32 human antibody preferably comprises the Fab fragment or the scFv fragment of A32. The antigen binding portion of A32 most preferably comprises a light chain amino acid sequence of SEQ ID NO: 1, which is encoded by the nucleic acid sequence of SEQ ID NO: 2, and a heavy chain amino acid sequence of SEQ ID NO: 3, which is encoded by the nucleic acid sequence of SEQ ID NO: 4. The generation of antibody fragments can be accomplished using routine molecular biology techniques that are within the skill of an ordinary artisan. While the fusion protein preferably comprises a light chain and a heavy chain of an A32 human antibody, or variants or fragments thereof, the fusion protein also can comprise a single chain of the A32 antibody (i.e., a light chain or a heavy chain). In this embodiment, the nucleic acid sequence encoding the A32 antibody chain that is not included in the fusion protein can be co-expressed with the nucleic acid sequence encoding the fusion protein, both of which can then be assembled into a larger protein molecule using routine molecular biology techniques. Alternatively, the A32 antibody chain that is not included in the fusion protein can be co-administered with the fusion protein to the mammal.

In one embodiment of the invention, the fusion protein further comprises an antigen-binding portion of a second antibody, or a variant thereof. The second antibody to be used in the inventive method preferably is broadly cross-reactive (e.g., can bind to a broad range of viral primary isolates from different strains and clades) with a high neutralization activity (e.g., typically with an IC₅₀ of less than 100 μg/ml). The second antibody binds to an epitope of an envelope protein of HIV that is exposed upon HIV binding to a CD4 receptor (i.e., a CD4-induced (CD4i) antibody). The second antibody can be any suitable CD4i antibody. Examples of suitable CD4i antibodies include 17b, 48d, Fab X5, m12, m6, and m9. The second antibody preferably is an m9 antibody. The m9 antibody is an scFv fragment derived from Fab X5 by random mutagenesis and sequential antigen panning, which exhibits potent neutralization of a broad range of primary HIV-1 isolates (see, e.g., Zhang et al., J. Mol. Biol., 335, 209-219 (2004)). An exemplary fusion protein comprising an scFv fragment of the A32 antibody and an antigen binding portion of the m9 antibody has an amino acid sequence of SEQ ID NO: 5. While the second antibody can bind to an epitope of any envelope protein of HIV, preferably it binds to an epitope of the gp120 envelope protein, which, as discussed above, mediates cell entry by binding to a CD4 receptor. Thus, in this embodiment, the A32 portion of the fusion protein induces conformational changes in the structure of gp120 that exposes a CD4-induced epitope recognized by the second antibody portion of the fusion protein, thereby enhancing the efficiency with which the second antibody neutralizes HIV-1 isolates.

In another embodiment of the invention, the fusion protein further comprises an immunogenic portion of an envelope protein of HIV, or a variant thereof. By an “immunogenic portion” is meant any portion of an HIV envelope protein that induces a measurable immune response in a suitable host, and also is referred to as an “epitope.” An “immune response” can entail, for example, antibody production and/or the activation of immune effector cells. The HIV envelope (Env) protein is a glycoprotein complex comprising two subunits: gp120 and gp41. Thus, the fusion protein can comprise an immunogenic portion of gp120 and/or gp41. In a preferred embodiment of the invention, the fusion protein comprises an immunogenic portion of a gp120 protein, or a variant thereof. The fusion protein can comprise any suitable immunogenic portion of gp120. The principal virus-neutralizing epitope of gp120 is located within a hypervariable loop in the third variable domain (V3) of gp120 (see, e.g., Goudsmit et al., Proc. Natl. Acad. Sci. USA, 85, 4478-4482 (1988), Palker et al., Proc. Natl. Acad. Sci. USA, 85, 1932-1933 (1988), Javaherian et al., Proc. Natl. Acad. Sci. USA, 86, 6768-6772 (1989), and Gorny et al., J. Virol., 78, 2394-2404 (2004)). Thus, the immunogenic portion of an HIV envelope protein preferably comprises the V3 domain of gp120. The V3 domain of gp120, however, is merely an exemplary immunogenic portion of gp120, and other immunogenic portions of gp120, including the entire gp120 polypeptide, can be used in connection with the inventive fusion protein.

In yet another embodiment of the invention, the fusion protein can further comprise a soluble CD4 (sCD4) polypeptide capable of binding to HIV, or a variant thereof. Soluble forms of CD4 have been shown to inhibit HIV infection (see, e.g., Deen et al., Nature, 331, 82-84 (1988), and Fisher et al., Nature, 331, 76-78 (1988)). Soluble CD4 binding to gp120 binding enhances the binding of A32 to gp120 (see, e.g., Wyatt et al., supra), suggesting that sCD4-gp120 binding enhances the exposure of the A32 epitope on gp120. Any suitable sCD4 polypeptide can be used in the inventive method. Suitable sCD4 polypeptides are known in the art and are available commercially from, for example, ImmunoDiagnostics, Inc. (Woburn, Mass.) and Protein Sciences Corp. (Meriden, Conn.).

The inventive fusion protein comprising an antigen binding portion of an A32 antibody can comprise either an antigen-binding portion of a second antibody, an immunogenic portion of an HIV envelope protein, or a soluble CD4 polypeptide. In this respect, the fusion protein can comprise an antigen-binding portion of one or more second antibodies (i.e., a second, third, and fourth antibody), one or more immunogenic portions of an HIV envelope protein, or one or more soluble CD4 polypeptides. Alternatively, to maximize the immune response against HIV infection, the inventive fusion protein comprising an antigen binding portion of an A32 antibody can comprise an antigen-binding portion of a second antibody, an immunogenic portion of an HIV envelope protein, and/or a soluble CD4 polypeptide in any suitable combination. Thus, for example, the fusion protein can comprise an antigen binding portion of A32, or a variant thereof, and an antigen-binding portion of a second antibody that binds to an epitope of an HIV envelope protein that is exposed upon HIV binding to CD4, and a soluble CD4 polypeptide capable of binding to HIV. In this embodiment, the fusion protein preferably comprises an antigen binding portion of A32, an antigen binding portion of the m9 antibody, and a sCD4 polypeptide capable of binding to HIV (an A32-m9-sCD4 fusion protein).

The inventive fusion protein can be generated using routine molecular biology techniques, such as restriction enzyme or recombinational cloning techniques (see, e.g., Gateway™ (Invitrogen) and U.S. Pat. Nos. 5,314,995 and 5,994,104). In one embodiment, the polypeptide components of the inventive fusion protein can be joined together by a long flexible linker. The linker can be any suitable long flexible linker, such that the fusion protein can bind to the epitope of the viral envelope protein (i.e., the fusion protein is not excluded from binding by molecular steric hindrance). The linker can be any suitable length, but is preferably at least about 15 (e.g., at least about 20, at least about 25, at least about 30, at least about 35, at least about 40, at least about 45, at least about 50, or ranges thereof) amino acids in length. Preferably, the long flexible linker is an amino acid sequence that is naturally present in immunoglobulin molecules of the host, such that the presence of the linker would not result in an immune response against the linker sequence by the mammal.

The inventive fusion protein also can include additional peptide sequences which act to promote stability, purification, and/or detection of the fusion protein. For example, a reporter peptide portion (e.g., green fluorescent protein (GFP), β-galactosidase, or a detectable domain thereof) can be incorporated in the fusion protein. Purification-facilitating peptide sequences include those derived or obtained from maltose binding protein (MBP), glutathione-S-transferase (GST), or thioredoxin (TRX). The fusion protein also or alternatively can be tagged with an epitope which can be antibody purified (e.g., the Flag epitope, which is commercially available from Kodak (New Haven, Conn.)), a hexa-histidine peptide, such as the tag provided in a pQE vector available from QIAGEN, Inc. (Chatsworth, Calif.), or an HA tag (as described in, e.g., Wilson et al., Cell, 37, 767 (1984)).

Alternatively, the fusion protein can comprise a variant of the aforementioned antigen binding portion of an A32 human antibody, the antigen-binding portion of a second antibody, the immunogenic portion of an HIV envelope protein, and/or the soluble CD4 polypeptide. A variant of an antigen binding portion of the A32 human antibody desirably retains the ability to bind to the same epitope as an unmodified antigen binding portion of A32 (i.e., a gp120 epitope). A variant of the immunogenic portion of an HIV envelope protein desirably retains the ability to elicit a neutralizing antibody response against a broad range of HIV-1 isolates. A variant of the sCD4 polypeptide desirably retains the ability to recognize and bind to the same epitope of the HIV gp120 envelope protein as an unmodified sCD4 polypeptide. Such variants can be obtained by any suitable method, including random and site-directed mutagenesis of the nucleic acid encoding the relevant polypeptide (see, e.g., Walder et al., Gene, 42, 133-193 (1986), Bauer et al., Gene, 37, 73 (1985), U.S. Pat. Nos. 4,518,584 and 4,732,462, and QuikChange Site-Directed Mutagenesis Kit (Stratagene, LaJolla, Calif.)), and sequential antigen panning (see, e.g., International Patent Application Publication No. WO 03/092630). Variants also can be generated using codon optimization, in which codon frequency and/or codon pairs (i.e., codon context) are optimized for a particular species (e.g., humans, either by optimizing a non-human or human sequence by replacement of “rare” human codons based on codon frequency, such as by using techniques such as those described in Buckingham et al., Biochimie, 76(5), 351-54 (1994) and U.S. Pat. Nos. 5,082,767, 5,786,464, and 6,114,148).

While a variant of the nucleic acid encoding the relevant polypeptide component of the fusion protein can be generated in vivo and then isolated and purified, alternatively, a variant of the nucleic acid also can be synthesized. Various techniques used to synthesize nucleic acids are known in the art (see, e.g., Lemaitre et al., Proc. Natl. Acad. Sci., 84, 648-652 (1987)).

Additionally, a variant can be synthesized using peptide-synthesizing techniques known in the art (see, e.g., Bodansky, Principles of Peptide Synthesis, Springer-Verlag, Heidelberg, 1984). In particular, a variant can be synthesized using the procedure of solid-phase synthesis (see, e.g., Merrifield, J. Am. Chem. Soc., 85, 2149-54,(1963), Barany et al., Int. J. Peptide Protein Res., 30, 705-739 (1987), and U.S. Pat. No. 5,424,398). If desired, a variant can be synthesized with an automated peptide synthesizer. Removal of the t-butyloxycarbonyl (t-BOC) or 9-fluorenylmethyloxycarbonyl (Fmoc) amino acid blocking groups and separation of the polypeptide from the resin can be accomplished by, for example, acid treatment at reduced temperature. The variant-containing mixture can then be extracted, for instance, with dimethyl ether, to remove non-peptidic organic compounds, and the synthesized variant can be extracted from the resin powder (e.g., with about 25% w/v acetic acid). Following the synthesis of the variant, further purification (e.g., using high performance liquid chromatography (HPLC)) optionally can be done in order to eliminate any incomplete polypeptides or free amino acids. Amino acid and/or HPLC analysis can be performed on the synthesized polypeptide to determine its identity. The variant can be produced as part of a larger fusion protein, such as by the above-described methods or genetic means, or as part of a larger conjugate, such as through physical or chemical conjugation.

The ability of a variant of the antigen binding portion of A32, or a variant of the antigen binding portion of the second antibody, to bind to the same epitope as an unmodified A32 antibody or an unmodified second antibody can be assessed by any suitable manner known in the art, such as by enzyme-linked immunosorbent assay (ELISA). A variant includes molecules that have about 50% or more amino acid identity to the polypeptide of interest (e.g., an antigen binding portion of A32). The variant preferably includes molecules that have about 75% amino acid identity to the polypeptide of interest. The variant more preferably includes molecules that have about 85% (e.g., about 90% or more, about 95% or more, about 96% or more, about 97% or more, about 98% or more, or about 99% or more) amino acid identity with the polypeptide of interest. The degree of amino acid identity can be determined using any method known in the art, such as the BLAST sequence database. Ideally, the variant contains from 1 to about 40 (e.g., about 5, about 10, about 15, about 20, about 25, about 30, about 35, or ranges thereof) amino acid substitutions, deletions, inversions, and/or insertions thereof. The variant more preferably contains from 1 to about 20 amino acid substitutions, deletions, inversions, and/or insertions thereof. The variant most preferably contains from 1 to about 10 amino acid substitutions, deletions, inversions, and/or insertions thereof.

The substitutions, deletions, inversion, and/or insertions of the antigen binding portion of A32, the antigen-binding portion of a second antibody, the immunogenic portion of an HIV envelope protein, and/or the soluble CD4 polypeptide, to produce variants thereof preferably occur in non-essential regions of the respective polypeptide. An “essential” amino acid sequence is one that is required for normal function of the polypeptide comprising the amino acid sequence The identification of essential and non-essential amino acids can be achieved by methods known in the art, such as by site-directed mutagenesis and AlaScan analysis (see, e.g., Moffison et al., Chem. Biol. 5(3), 302-307 (2001)). Essential amino acids desirably are maintained or replaced by conservative substitutions in the variants, such that, for example, the antigen binding portion of A32 maintains the ability to bind to an epitope of an HIV gp120 envelope protein. Non-essential amino acids can be deleted, or replaced by a spacer or by conservative or non-conservative substitutions.

The variants can be obtained by substitution of any of the amino acids as present in the polypeptide of interest. As can be appreciated, there are positions in a particular polypeptide sequence that are more tolerant to substitutions than others, and some substitutions can improve the function of the polypeptide (e.g., the binding activity of the native antigen binding portion of A32). The essential amino acids should either not be substituted, or be substituted with conservative amino acid substitutions. The amino acids that are nonessential can either not be substituted, can be substituted by conservative or non-conservative substitutions, and/or can be deleted.

Conservative substitution refers to the replacement of an amino acid with a naturally or non-naturally occurring amino acid having similar steric properties. Where the side-chain of the amino acid to be replaced is either polar or hydrophobic, the conservative substitution should be with a naturally or non-naturally occurring amino acid that is also polar or hydrophobic (in addition to having the same steric properties as the side-chain of the replaced amino acid). When the native amino acid to be replaced is charged, the conservative substitution can be with a naturally or non-naturally occurring amino acid that is charged, or with a non-charged (polar, hydrophobic) amino acid that has the same steric properties as the side-chains of the replaced amino acid. For example, the replacement of arginine by glutamine, aspartate by asparagine, or glutamate by glutamine is considered to be a conservative substitution.

In order to further exemplify what is meant by conservative substitution, Groups A-F are listed below. The replacement of one member of the following groups by another member of the same group is considered to be a conservative substitution.

Group A includes leucine, isoleucine, valine, methionine, phenylalanine, serine, cysteine, threonine, and modified amino acids having the following side chains: ethyl, iso-butyl, —CH₂CH₂OH, —CH₂CH₂CH₂OH, —CH₂CHOHCH₃ and CH₂SCH₃.

Group B includes glycine, alanine, valine, serine, cysteine, threonine, and a modified amino acid having an ethyl side chain.

Group C includes phenylalanine, phenylglycine, tyrosine, tryptophan, cyclohexylmethyl, and modified amino residues having substituted benzyl or phenyl side chains.

Group D includes glutamic acid, aspartic acid, a substituted or unsubstituted aliphatic, aromatic or benzylic ester of glutamic or aspartic acid (e.g., methyl, ethyl, n-propyl, iso-propyl, cyclohexyl, benzyl, or substituted benzyl), glutamine, asparagine, CO—NH-alkylated glutamine or asparagine (e.g., methyl, ethyl, n-propyl, and iso-propyl), and modified amino acids having the side chain —(CH₂)₃COOH, an ester thereof (substituted or unsubstituted aliphatic, aromatic, or benzylic ester), an amide thereof, and a substituted or unsubstituted N-alkylated amide thereof.

Group E includes histidine, lysine, arginine, N-nitroarginine, p-cycloarginine, g-hydroxyarginine, N-amidinocitruline, 2-amino guanidinobutanoic acid, homologs of lysine, homologs of arginine, and ornithine.

Group F includes serine; threonine, cysteine, and modified amino acids having C₁-C₅ straight or branched alkyl side chains substituted with —OH or —SH.

A non-conservative substitution is a substitution in which the substituting amino acid (naturally or non-naturally occurring) has a significantly different size, configuration and/or electronic properties compared with the amino acid being substituted. Thus, the side chain of the substituting amino acid can be significantly larger (or smaller) than the side chain of the native amino acid being, substituted and/or can have functional groups with significantly different electronic properties than the amino acid being substituted. Examples of non-conservative substitutions of this type include the substitution of phenylalanine or cycohexylmethyl glycine for alanine, or isoleucine for glycine. Alternatively, a functional group can be added to the side chain, deleted from the side chain or exchanged with another functional group. Examples of nonconservative substitutions of this type include adding an amine, hydroxyl, or carboxylic acid to the aliphatic side chain of valine, leucine or isoleucine, or exchanging the carboxylic acid in the side chain of aspartic acid or glutamic acid with an amine or deleting the amine group in the side chain of lysine or ornithine.

For non-conservative substitutions, the side chain of the substituting amino acid can have significantly different steric and electronic properties from the functional group of the amino acid being substituted. Examples of such modifications include tryptophan for glycine, and lysine for aspartic acid.

The inventive fusion molecule, such as an A32-m9-sCD4 fusion protein preferably recognizes and binds to one or more strains of HIV. For example, the fusion protein preferably recognizes and binds to an epitope of a viral envelope protein of HIV-1 and HIV-2. The fusion protein also is preferably broadly cross-reactive (e.g., can bind to a wide range of isolates from different clades). For example, the fusion protein preferably binds to an epitope of a viral envelope protein of two, three, four, five, six, seven, or each of the clades selected from the group consisting of A, B, C, D, E, EA, F, and G.

The invention further provides a nucleic acid molecule encoding the above-described fusion protein. “Nucleic acid molecule” is intended to encompass a polymer of DNA or RNA, i.e., a polynucleotide, which can be single-stranded or double-stranded and which can contain non-natural or altered nucleotides. In one embodiment, the nucleic acid molecule can lack introns or portions thereof. The nucleic acid molecule preferably is DNA. The nucleic acid molecule may be isolated or purified from any suitable source. For example, the nucleic acid molecule may be isolated or purified from tissues or chemically synthesized by methods known in the art. With respect to the antigen binding portion of the A32 human antibody, the light chain amino acid sequence preferably is encoded by the nucleic acid sequence of SEQ ID NO: 2, and the heavy chain amino acid sequence preferably is encoded by the nucleic acid sequence of SEQ ID NO: 4.

The invention provides a method of inhibiting a viral infection in a mammal, which method comprises administering to a mammal in need thereof an effective amount of the aforementioned fusion protein. An “effective amount” means an amount sufficient to show a meaningful benefit in an individual, e.g., promoting at least one aspect of HIV treatment, prevention, or amelioration of other relevant medical condition(s) associated with HIV infection. Effective amounts may vary depending upon the individual and/or the specific characteristics of the fusion protein. The fusion protein can be administered to any suitable mammal, but preferably is administered to a human. The fusion protein can be administered to a mammal as an amino acid molecule, as a nucleic acid molecule encoding the fusion protein, as a vector comprising the nucleic acid molecule encoding the fusion protein, or as a cell (e.g., a host cell) comprising any of the above.

When the fusion protein is administered to a mammal as a vector comprising a nucleic acid molecule encoding the fusion protein, any suitable vector can be used in this context. Suitable vectors include nucleic acid vectors, such as naked DNA and plasmids, liposomes, molecular conjugates, and viral vectors, such as retroviral vectors, parvovirus-based vectors (e.g., adenoviral-based vectors and adeno-associated virus (AAV)-based vectors), lentiviral vectors (e.g., Herpes simplex (HSV)-based vectors), and hybrid or chimeric viral vectors, such as an adenoviral backbone with lentiviral components (see, e.g., Zheng et al., Nat. Biotech., 18(2), 176-80 (2000); International Patent Application WO 98/22143; International Patent Application WO 98/46778; and International Patent Application WO 00/17376) and an adenoviral backbone with AAV components (see, e.g., Fisher et al., Hum. Gene Ther., 7, 2079-2087 (1996)). Vectors and vector construction are known in the art (see, e.g., Sambrook et al., Molecular Cloning: A Laboratory Manual, 2d edition, Cold Spring Harbor Laboratory, NY (1989); and Ausubel et al., Current Protocols in Molecular Biology, Green Publishing Associates and John Wiley & Sons, New York, N.Y. (1994)).

The vector can comprise any suitable promoter and other regulatory sequences (e.g., transcription and translation initiation and termination codons) to control the expression of the nucleic acid sequence encoding the fusion protein. The promoter can be a native or nonnative promoter operably linked to the nucleic acid molecule described above. The selection of promoters, including various constitutive and regulatable promoters, is within the skill of an ordinary artisan. Examples of regulatable promoters include inducible, repressible, and tissue-specific promoters. Specific examples include viral promoters, such as adenoviral promoters, AAV promoters, and CMV promoters. Additionally, operably linking the nucleic acid described above to a promoter is within the skill in the art.

The fusion protein can be administered to a mammal in the form of a cell comprising a nucleic acid sequence encoding the fusion protein, optionally in the form of a vector. Thus, the invention also provides an isolated or purified cell comprising a vector or nucleic acid molecule encoding the fusion protein, from which the fusion protein desirably is secreted. In this embodiment, any suitable cell (e.g., an isolated cell) can be used. Examples include host cells, such as E. coli (e.g., E. coli Th-1, TG-2, DH5α, XL-Blue MRF′ (Stratagene), SA2821, and Y1090), Bacillus subtilis, Salmonella typhinurium, Serratia marcescens, Pseudomonas (e.g., P. aerugenosa), N. grassa, insect cells (e.g., Sf9, Ea4), yeast (S. cerevisiae) cells, and cells derived from a mammal, including human cell lines. Specific examples of suitable eukaryotic cells include VERO, HeLa, 3T3, Chinese hamster ovary (CHO) cells, W138 BHK, COS-7, and MDCK cells. Alternatively and preferably, cells from a mammal, such as a human, to be treated in accordance with the methods described herein can be used as host cells. In a preferred embodiment, the cell is a human B cell. Methods of introducing vectors into isolated host cells and the culture and selection of transformed host cells in vitro are known in the art and include the use of calcium chloride-mediated transformation, transduction, conjugation, triparental mating, DEAE, dextran-mediated transfection, infection, membrane fusion with liposomes, high velocity bombardment with DNA-coated microprojectiles, direct microinjection into single cells, and electroporation (see, e.g., Sambrook et al., supra, Davis et al., Basic Methods in Molecular Biology (1986), and Neumann et al., EMBO J. 1, 841 (1982)). Desirably, the cell comprising the vector or nucleic acid sequence expresses the nucleic acid sequence encoding the fusion protein, such that the nucleic acid sequence is transcribed and translated efficiently by the cell.

The nucleic acid molecule, cell, vector, or fusion protein can be administered to any mammal in need thereof. The fusion protein preferably is administered to a human. As a result of administration of the fusion protein to the mammal, infection of the mammal by HIV is inhibited. The inventive method can inhibit infection by any type of HIV, but preferably inhibits HIV-1 and/or HIV-2 infection. The inventive fusion protein also is preferably broadly cross-reactive. Thus, the inventive method can be used to inhibit infection by any HIV group (e.g., groups M and/or O), and subtype (e.g., clades A, B, C, D, E, EA, F, and/or G).

The nucleic acid molecules, vectors, cells, and fusion proteins can be administered to a mammal alone, or in combination with a pharmaceutically acceptable carrier. By pharmaceutically acceptable is meant a material that is not biologically or otherwise undesirable (i.e., the material can be administered to a mammal, along with the nucleic acid, vector, cell, or fusion protein, without causing any undesirable biological effects or interacting in a deleterious manner with any of the other components of the pharmaceutical composition in which it is contained). The carrier is selected to minimize any degradation of the fusion protein and to minimize any adverse side effects in the mammal as would be well-known to one of ordinary skill in the art.

Suitable carriers and their formulations are described in A. R. Gennaro, ed., Remington: The Science and Practice of Pharmacy (19th ed.), Mack Publishing Company, Easton, Pa. (1995). Pharmaceutical carriers, include sterile water, saline, Ringer's solution, dextrose solution, and buffered solutions at physiological pH. Typically, an appropriate amount of a pharmaceutically acceptable salt is used in the formulation to render the formulation isotonic. The pH of the formulation is preferably from about 5 to about 8 (e.g., about 5.5, about 6, about 6.5, about 7, about 7.5, and ranges thereof). More preferably, the pH is about 7 to about 7.5. Further carriers include sustained-release preparations, such as semipermeable matrices of solid hydrophobic polymers containing the fusion protein, which matrices are in the form of shaped articles (e.g., films, liposomes, or microparticles). It will be apparent to those persons skilled in the art that certain carriers may be more preferable depending upon, for instance, the route of administration and concentration of composition being administered.

Compositions (e.g., pharmaceutical compositions) comprising the nucleic acid molecule, vector, cell, or fusion protein can include carriers, thickeners, diluents, buffers, preservatives, surface active agents and the like. The compositions can also include one or more active ingredients, such as antimicrobial agents, anti-inflammatory agents, anesthetics, and the like.

The composition (e.g., pharmaceutical composition) comprising the nucleic acid molecule, vector, cell, or fusion protein can be administered in any suitable manner depending on whether local or systemic treatment is desired, and on the area to be treated. Administration can be topical (including ophthalmical, vaginal, rectal, intranasal, transdermal, and the like), oral, by inhalation, or parenteral (including by intravenous drip or subcutaneous, intracavity, intraperitoneal, or intramuscular injection). Topical intranasal administration refers to the delivery of the compositions into the nose and nasal passages through one or both of the nares and can comprise delivery by a spraying mechanism or droplet mechanism, or through aerosolization of the nucleic acid, vector, or fusion protein. Administration of the compositions by inhalant can be through the nose or mouth via delivery by a spraying or droplet mechanism. Delivery can also be directly to any area of the respiratory system (e.g., lungs) via intubation.

If the composition is to be administered parenterally, the administration is generally by injection. Injectables can be prepared in conventional forms, either as liquid solutions or suspensions, solid forms suitable for suspension in liquid prior to injection, or as emulsions. Additionally, parental administration can involve the preparation of a slow-release or sustained-release system, such that a constant dosage is maintained. Preparations for parenteral administration include sterile aqueous or non-aqueous solutions, suspensions, and emulsions. Examples of non-aqueous solvents are propylene glycol, polyethylene glycol, vegetable oils, such as olive oil, and injectable organic esters, such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives also can be present such as, for example, antimicrobials, anti-oxidants, chelating agents, and inert gases and the like.

Formulations for topical administration may include ointments, lotions, creams, gels, drops, suppositories, sprays, liquids, and powders. Conventional pharmaceutical carriers; aqueous, powder, or oily bases; thickeners, and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable.

Some of the compositions can potentially be administered as a pharmaceutically acceptable acid- or base- addition salt, formed by reaction with inorganic acids, such as hydrochloric acid, hydrobromic acid; perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base, such as sodium hydroxide, ammonium hydroxide, potassium hydroxide, and organic bases, such as mono-, di-, trialkyl, and aryl amines and substituted ethanolamines.

The nucleic acid molecule, vector, or fusion protein can be administered with a pharmaceutically acceptable carrier and can be delivered to the mammal's cells in vivo and/or ex vivo by a variety of mechanisms well-known in the art (e.g., uptake of naked DNA, liposome fusion, intramuscular injection of DNA via a gene gun, endocytosis, and the like).

Additionally, probiotic therapies are envisioned by the present invention. Viable host cells containing the nucleic acid or vector of the invention and expressing the fusion protein can be used directly as the delivery vehicle for the fusion protein to the desired site(s) in vivo. Preferred host cells for the delivery of the fusion protein directly to desired site(s), such as, for example, to a selected body cavity, can comprise bacteria. More specifically, such host cells can comprise suitably engineered strain(s) of lactobacilli, enterococci, or other common bacteria, such as E. coli, normal strains of which are known to commonly populate body cavities. More specifically yet, such host cells can comprise one or more selected nonpathogenic strains of lactobacilli, such as those described by Andreu et al., J. Infect. Dis., 171(5), 1237-43 (1995), especially those having high adherence properties to epithelial cells (e.g., vaginal epithelial cells) and suitably transformed using the nucleic acid or vector of the invention.

If ex vivo methods are employed, cells or tissues can be removed and maintained outside the body according to standard protocols known in the art. The compositions can be introduced into the cells via any gene transfer mechanism, such as calcium phosphate mediated gene delivery, electroporation, microinjection, or proteoliposomes. The transduced cells then can be infused (e.g., with a pharmaceutically acceptable carrier) or homotopically transplanted back into the mammal per standard methods for the cell or tissue type. Standard methods are known for transplantation or infusion of various cells into a mammal.

The exact amount of the composition required to treat an HIV infection will vary from mammal to mammal, depending on the species, age, gender, weight, and general condition of the mammal, the nature of the virus, the existence and extent of viral infection, the particular fusion proteins, nucleic acid, vector, or cell used, the route of administration, and whether other drugs are included in the regimen. Thus, it is not possible to specify an exact amount for every composition. However, an appropriate amount can be determined by one of ordinary skill in the art using only routine experimentation given the teachings herein. Effective dosages and schedules for administering the nucleic acid molecules, vectors, cells, and fusion proteins of the invention can be determined empirically, and making such determinations is within the skill in the art. The dosage ranges for the administration of the compositions are those large enough to produce the desired effect; however, the dosage should not be so large as to cause adverse side effects, such as unwanted cross-reactions, anaphylactic reactions, and the like. Dosage can vary, and can be administered in one or more (e.g., two or more, three or more, four or more, or five or more) doses daily, for one or more days. The composition can be administered before HIV infection or immediately upon determination of HIV infection and continuously administered until the virus is undetectable.

A typical daily dosage of the fusion protein might range from about 1 μg/kg to up to 100 mg/kg of body weight or more per day, depending on the factors mentioned above. For example, the range can be from about 100 mg to one gram per dose. Nucleic acids, vectors, and host cells should be administered so as to result in comparable levels of production of fusion molecules.

The fusion protein of the invention can be combined with other well-known HIV therapies, and prophylactic vaccines already in use. The combination of the fusion protein of the invention can generate an additive or a synergistic effect with current treatments. The fusion protein of the invention can be combined with other HIV and AIDS therapies and vaccines, such as highly active antiretroviral therapy (HAART), which comprises a combination of protease inhibitors and reverse transcriptase inhibitors, azidothymidine (AZT), structured treatment interruptions of HAART, cytokine immune enhancement therapy (e.g., interleukin (IL)-2, IL-12, CD40L+IL-12, IL-7, HIV protease inhibitors (e.g., ritonavir, indinavir, and nelfinavir, etc.), and interferons (IFNs)), cell replacement therapy, recombinant viral vector vaccines, DNA vaccines, inactivated virus preparations, immunosuppressive agents, such as Cyclosporin A, and cyanovirin therapy (see, e.g., U.S. Pat. No. 6,015,876 and International Patent Application Publication No. WO 03/072594). Such therapies can be administered in the manner already in use for the known treatment providing a therapeutic or prophylactic effect (see, e.g., Silvestri et al. Immune Intervention in AIDS. In: Immunology of Infectious Disease, H. E. Kauffman, A. Sher, and R. Ahmed eds., ASM Press, Washington D.C. (2002)).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A fusion protein comprising an antigen binding portion of an A32 human antibody, or variant thereof, and one of the following:
(a) an antigen-binding portion of a second antibody, or a variant thereof, wherein the second antibody binds to an epitope of an envelope protein of a human immunodeficiency virus (HIV) that is exposed upon the HIV binding to a CD4 receptor,
(b) an immunogenic portion of an envelope protein of HIV, or a variant thereof, or
(c) a soluble CD4 (sCD4) polypeptide capable of binding to HIV, or a variant thereof.

2. The fusion protein of claim 1, wherein the portion of the A32 human antibody comprises a light chain amino acid sequence of SEQ ID NO: 1, or a variant thereof.

3. The fusion protein of claim 2, wherein the light chain amino acid sequence is encoded by the nucleic acid sequence of SEQ ID NO: 2.

4. The fusion protein of claim 1, wherein the portion of the A32 human antibody comprises a heavy chain amino acid sequence of SEQ ID NO: 3, or a variant thereof.

5. The fusion protein of claim 4, wherein the heavy chain amino acid sequence is encoded by the nucleic acid sequence of SEQ ID NO: 4.

6. The fusion protein of claim 1, wherein the fusion protein comprises an antigen-binding portion of a second antibody, or variant thereof, wherein the antigen-binding portion of the second antibody binds to an epitope of an envelope protein of HIV that is exposed upon HIV binding to a CD4 receptor.

7. The fusion protein of claim 6, wherein the second antibody is an m9 antibody.

8. The fusion protein of claim 6, wherein the epitope of the envelope protein is an epitope of HIV glycoprotein 120 (gp120).

9. The fusion protein of claim 6, wherein the fusion protein comprises SEQ ID NO: 5.

10. The fusion protein of claim 1, wherein the fusion protein comprises an immunogenic portion of an envelope protein of the HIV, or a variant thereof.

11. The fusion protein of claim 10, wherein the envelope protein is an immunogenic portion of HIV glycoprotein 120 (gp120), or a variant thereof.

12. The fusion protein of claim 1, wherein the fusion protein comprises at least one antigen-binding portion of a second antibody, or variant thereof, and a soluble CD4 polypeptide capable of binding to HIV, or variant thereof, wherein the second antibody binds to an epitope of an envelope protein of the HIV that is exposed upon the HIV binding to a CD4 receptor.

13. The fusion protein of claim 12, wherein the second antibody is an m9 antibody.

14. The fusion protein of claim 1, wherein the fusion protein binds with HIV.

15. The fusion protein of claim 14, wherein the fusion protein can bind more than one clade of HIV.

16. A nucleic acid molecule comprising a nucleic acid sequence encoding the fusion protein of claim 1.

17. An isolated or purified cell comprising a vector or nucleic acid molecule encoding the fusion protein of claim 1.

18. The isolated or purified cell of claim 17, wherein the cell is a B cell.

19. The isolated or purified cell of claim 17, wherein the cell secretes the fusion protein.

20. A method of inhibiting an HIV infection in a mammal, which method comprises administering to a mammal in need thereof an effective amount of the fusion protein of claim 1.

21. The method of claim 20, wherein the mammal is a human.

22. A method of inhibiting an HIV infection in a mammal, which method comprises administering to a mammal in need thereof an effective amount of the nucleic acid sequence of claim 16, optionally in the form of a vector, wherein the nucleic acid sequence or vector is optionally contained within a host cell.

23. The method of claim 22, wherein the mammal is a human.

24. A fusion protein comprising (i) a light chain amino acid sequence of an A32 human antibody, or a variant thereof, or a heavy chain amino acid sequence of an A32 human antibody, or a variant thereof, and (ii) one of the following:
(a) an antigen-binding portion of a second antibody, or a variant thereof, wherein the second antibody binds to an epitope of an envelope protein of a human immunodeficiency virus (HIV) that is exposed upon the HIV binding to a CD4 receptor,
(b) an immunogenic portion of an envelope protein of HIV, or a variant thereof, or
(c) a soluble CD4 (sCD4) polypeptide capable of binding to HIV, or a variant thereof.

25. The fusion protein of claim 24, wherein the light chain amino acid sequence of the A32 human antibody comprises SEQ ID NO: 1, or a variant thereof.

26. The fusion protein of claim 25, wherein the light chain amino acid sequence of the A32 human antibody is encoded by the nucleic acid sequence of SEQ ID NO: 2.

27. The fusion protein of claim 24, wherein the heavy chain amino acid sequence of the A32 human antibody comprises SEQ ID NO: 3, or a variant thereof.

28. The fusion protein of claim 27, wherein the heavy chain amino acid sequence of the A32 human antibody is encoded by the nucleic acid sequence of SEQ ID NO: 4.
